(19) United States
(12) Patent Application Publication
(10) Pub. No.: US 2024/0074855 A1
(43) Pub. Date: Mar. 7, 2024

(54) EXPANDABLE PROSTHETIC HEART VALVES

(71) Applicant: EDWARDS LIFESCIENCES CORPORATION, Irvine, CA (US)

(72) Inventors: Eitan Atias, Netanya (IL); Elazar Levi Schwarcz, Netanya (IL); Danny M. Garmahi, Hadera (IL)

(73) Assignee: EDWARDS LIFESCIENCES CORPORATION, Irvine, CA (US)

(21) Appl. No.: 18/372,607

(22) Filed: Sep. 25, 2023

Related U.S. Application Data
(63) Continuation of application No. PCT/US2022/026113, filed on Apr. 25, 2022.
(60) Provisional application No. 63/179,766, filed on Apr. 26, 2021.

(51) Int. Cl. A61F 2/24

(52) U.S. Cl. CPC A61F 2/243; A61F 2/2436; A61F 2/2439; A61F 2250/001

(57) ABSTRACT

This disclosure is directed to prosthetic heart valves having expansion and locking assemblies. As one example, a prosthetic heart valve can include an annular frame and an expansion and locking assembly that is configured to radially expand the frame to a radially expanded state and/or to lock the prosthetic valve in the radially expanded state to prevent the prosthetic valve from collapsing (i.e., radially compressing). In some examples, the prosthetic heart valve can be configured to radially self-expand to a partially radially expanded state, and can then be further radially expanded to the radially expanded state and/or locked in the radially expanded state by pulling an actuator member of the expansion and locking assembly. In some examples, the actuator member can extend through openings in vertical struts of the frame.

### CROSS-REFERENCE TO RELATED APPLICATIONS

The present application is a continuation of International Application No. PCT/US2022/026113, filed Apr. 25, 2022, which claims the benefit of U.S. Application No. 63/179,766, filed Apr. 26, 2021, both of which applications are incorporated herein by reference.

### FIELD

The present disclosure relates to expandable prosthetic heart valves, and to methods, assemblies, and apparatuses for delivering and deploying (e.g., positioning, radially expanding, locking, etc.,) such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery assembly and advanced through the patient's vasculature (e.g., through a femoral artery and the aorta) until the prosthetic heart valve reaches the implantation site in the heart.

Once at the implantation site, the prosthetic heart valve can be expanded to its functional size by, for example, actuating a mechanical actuator that applies a radial outward force to the prosthetic heart valve. Such prosthetic heart valves (ones that rely on a mechanical actuator for expansion) can be referred to as “mechanically expandable” prosthetic heart valves.

In other examples, the prosthetic heart valve can be self-expanding. For example, such self-expanding valves can be constructed from a shape-memory material and can be pre-formed in a radially expanded state so that the prosthetic valves are naturally biased to this radially expanded state. Such self-expanding valves can be held in a crimped state by a restraint (e.g., sheath, lasso, etc.) during delivery, but can be released from this restraint once at the implantation site so that they can self-expand to their functional size. For example, a user can push the self-expanding valve out of a delivery sheath by, for example, actuating a pushing mechanism of the delivery assembly.

However, even for self-expanding valves, it can be desirable to further expand the prosthetic valves with a mechanical actuator. And, for both self-expanding and mechanically expanding valves, it can be desirable to include a locking mechanism that holds the prosthetic valve in a radially expanded state in order to prevent collapse of the prosthetic valve. However existing mechanical actuators and locking mechanisms can be bulky, complicated to use, and/or costly to manufacture/produce. Thus, there remains a need for transcatheter prosthetic heart valves with improved expansion and locking mechanisms.

### SUMMARY

The present disclosure relates to expandable prosthetic heart valves, and to methods, assemblies, and apparatuses for delivering, expanding, locking, implanting, and deploying such prosthetic heart valves.

In one representative example, a prosthetic heart valve comprises an annular frame, an actuator member, and a locking element. The annular frame comprises an inflow end and an outflow end, and is radially compressible and expandable between a radially compressed state and a radially expanded state. Further, the frame is configured to radially self-expand from the radially compressed state to at least a partially radially expanded state. The actuator member is coupled to the frame at first and second axially spaced locations and is configured to apply a proximally directed force to the frame to radially expand and/or lock the prosthetic heart valve. The locking element is coupled to the frame at the second location and is configured to allow the actuator member to slide in only a proximal direction relative to the frame. The locking element also is configured to continuously engage the actuator member to prevent the actuator member from sliding in an opposite distal direction past the locking element.

In another representative example, a prosthetic heart valve, comprises an annular frame, a skirt assembly, an actuator member, and a locking element. The annular frame comprises an inflow end and an outflow end, and is radially compressible and expandable between a radially compressed state and a radially expanded state. Further, the frame is configured to radially self-expand to at least a partially expanded state when a restraining mechanism configured to hold the frame in the radially compressed state is loosened. The skirt assembly extends circumferentially around the frame and comprises a sleeve that is configured to receive the restraining mechanism. The actuator member is coupled to the frame at first and second axially spaced locations and is configured to apply a proximally directed force to the frame to radially expand and/or lock the prosthetic heart valve. The locking element is coupled to the frame at the second location and is configured to prevent radial compression of the valve when the actuator member is in a taut state by allowing the actuator member to slide in only a proximal direction relative to the frame and by continuously locking the actuator member to prevent the actuator member from sliding in an opposite distal direction past the locking element.

In yet another representative example, a prosthetic heart valve comprises an annular frame, a tension member, and a locking element. The annular frame comprises an inflow end and an outflow end, and is radially compressible and expandable between a radially compressed state and a radially expanded state. Further, the frame is configured to self-expand from the radially compressed state to at least a partially expanded state. The tension member is coupled to the frame at first and second axially spaced locations along the frame. The locking element is configured to engage the tension member at the second location and is configured to allow the tension member to be pulled proximally relative to the locking element and resist distal movement of the tension member relative to the locking element. Further, the locking element engages the tension member such that slack is present in the tension member when the frame self-expands to at least the partially expanded state. Application of a pulling force to the tension member is effective to remove the slack from the tension member to retain the frame in the at least partially expanded state.

In yet another representative example, a prosthetic heart valve comprises a tension member, a locking element, and an annular frame comprising first and second vertical struts. The annular frame comprises an inflow end and an outflow end, and is radially compressible and expandable between a radially compressed state and a radially expanded state. The annular frame includes the first vertical strut at an inflow end portion of the frame and includes the second vertical strut at an outflow end portion of the frame. The first vertical strut and/or the second vertical comprise one or more axially spaced openings that extend radially through the frame. The tension member is coupled to the first vertical strut and the second vertical strut and extends through the one or more axially spaced openings in the first vertical strut and/or the second vertical strut. The locking element is configured to engage the tension member at the second vertical strut and is configured to allow the tension member to be pulled proximally relative to the locking element and resist distal movement of the tension member relative to the locking element.

In yet another representative example, a method comprises loosening a restraining mechanism of a prosthetic heart valve to allow the prosthetic heart valve to radially self-expand; and pulling an actuator member of an expansion and locking assembly of the prosthetic heart valve a first distance to tighten a locking element of the expansion and locking assembly from a loose state to a taut state to lock the prosthetic heart valve and prevent the prosthetic heart valve from radially compressing.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a side elevation view of a delivery assembly for a prosthetic heart valve, according to one example.

FIG. 2 is side perspective view of a prosthetic heart valve and an expansion assembly for the prosthetic heart valve, according to one example, where the prosthetic valve is locked in a radially expanded state.

FIG. 3 is a top perspective view of the expansion assembly and the prosthetic heart valve of FIG. 2, where the prosthetic valve is in the radially expanded state.

FIG. 4 is a side perspective view of the expansion assembly and prosthetic heart valve of FIGS. 2-3, where the prosthetic valve is in the radially compressed state.

FIG. 5 is a side perspective view of the expansion assembly and the prosthetic heart valve of FIGS. 2-4, where the prosthetic valve is in a partially radially expanded state.

FIG. 6 is a side perspective view of the expansion assembly and the prosthetic heart valve of FIGS. 2-5, where the prosthetic valve is in the radially expanded state.

FIG. 7 is an enlarged view of a first portion of a frame of the prosthetic heart valve of FIGS. 2-6 that can contain an exemplary locking mechanism.

FIG. 8 is an enlarged view of a second portion of the frame of the prosthetic heart valve of FIGS. 2-6.

FIG. 9 is a top plan view of an outflow end of the first portion of the frame of the prosthetic heart valve shown in FIG. 7.

### DETAILED DESCRIPTION

### General Considerations

For purposes of this description, certain aspects, advantages, and novel features of the examples of this disclosure are described herein. The described methods, systems, and apparatus should not be construed as limiting in any way. Instead, the present disclosure is directed toward all novel and non-obvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Features, integers, characteristics, compounds, chemical moieties, or groups described in conjunction with a particular aspect, or example of the disclosure are to be understood to be applicable to any other aspect, or example described herein unless incompatible therewith. All of the features disclosed in this specification (including any accompanying claims, abstract, and drawings), and/or all of the steps of any method or process so disclosed, can be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed methods, systems, and apparatus can be used in conjunction with other systems, methods, and apparatus.

As used herein, the terms “a,” “an,” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

Directions and other relative references (e.g., inner, outer, upper, lower, etc.) can be used to facilitate discussion of the drawings and principles herein, but are not intended to be limiting. For example, certain terms can be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

As used herein, with reference to the prosthetic medical device (e.g., heart valve), capsule, and the delivery apparatus, “proximal” refers to a position, direction, or portion of a component that is closer to the user and/or a handle of the delivery apparatus that is outside the patient, while “distal” refers to a position, direction, or portion of a component that is further away from the user and/or the handle of the delivery apparatus and closer to the implantation site. The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the longitudinal axis of the prosthetic valve).

### The Disclosed Technology and Examples

Transcatheter prosthetic heart valves can be delivered in a crimped state to an implantation site on a delivery assembly (e.g., catheter). Once at the implantation site, the prosthetic valves can be expanded to their functional size (i.e., a radially expanded state) by releasing the prosthetic valves from a restraint (e.g., lasso, delivery sheath, etc.,) and allowing them to self-expand and/or by physically expanding the prosthetic valves via an expansion mechanism (e.g., mechanical actuator). The prosthetic valves also can be locked in the radially expanded state via a locking mechanism so that they do not subsequently collapse.

Self-expanding and mechanically expandable prosthetic heart valves can each provide their own sets of advantages and disadvantages. For example, the expansion of mechanically expandable valves can be relatively tightly controlled/regulated (e.g., the rate of expansion can be adjusted, expansion can be periodically paused and/or reversed, etc.,) whereas the expansion of self-expanding valves is generally less controllable. Specifically, mechanically expandable valves can be expanded to, and held in various working diameters (e.g., via a locking mechanism) and/or can be compressed after an initial expansion, allowing them to be repositioned and/or retrieved. However, actuators used in mechanically expandable valves, such as rotatable or push/pull actuators, are difficult to manufacture and can increase the overall crimp profile of the prosthetic valve. Conversely, self-expanding valves can be simpler to manufacture and can have a smaller crimp profile than mechanically expandable prosthetic heart valves. However, such self-expanding valve typically expand to their pre-set shape in one continuous motion that is generally difficult to control. Further, self-expandable valves may not be able to fully expand to their functional size when implanted in calcified native annulus, or once expanded, may not be able to retain their functional size under the radial compressive forces of the surrounding tissue.

Thus, both mechanically expandable and self-expanding prosthetic heart valves suffer from certain drawbacks.

Disclosed herein are prosthetic heart valves that can incorporate at least some of the benefits of both of these types of valves, without suffering from some or all of their drawbacks. For example, the prosthetic heart valves disclosed herein can be expanded and re-compressed in a more tightly controlled and regulated manner and can be locked in the expanded state (like mechanically expandable valves) but can have a smaller crimp profile and are easier to manufacture than known mechanically expandable valves.

Further, the prosthetic heart valves disclosed herein can be re-compressed (during assembly or during an implantation procedure) more easily than known mechanically expandable valves because, unlike some known prosthetic valves, the prosthetic valves disclosed herein can be re-compressed without having to first release/disengage a locking mechanism. As just one example, the prosthetic heart valves of the present disclosure can be at least partially self-expanding. Specifically, the prosthetic heart valves of the present disclosure can self-expand to a first radially expanded state (e.g., an intermediate radially expanded state), and then a physician can lock the prosthetic valves in the first radially expanded state and/or further mechanically expand the prosthetic valves to a second radially expanded state (e.g., a fully radially expanded state) via a mechanical actuator. However, because the prosthetic valves may not automatically lock in the first radially expanded state, the physician can easily re-compress the prosthetic valve (so that the prosthetic valve can be re-positioned within the native tissue to provide a better seal/fit with the native tissue), without having to release/disengage a locking mechanism.

Further, in some examples, the locking mechanisms of the present disclosure can be configured to continuously and/or automatically lock the prosthetic heart valves of the present disclosure at a range of valve diameters, allowing the physician to more smoothly expand the prosthetic heart valves as desired without having to worry about the prosthetic valves collapsing (i.e., retracting to a more radially compressed position). Additionally or alternatively, the expansion and locking assemblies disclosed herein can be simpler and/or easier to use, making the deployment process (e.g., the expansion and locking of the prosthetic valve) at the implantation site quicker and/or safer. As just one example, a physician can fully expand and/or lock the prosthetic valves disclosed herein by simply pulling a string, cord, cable, wire or other similar instrument. Further, due to the simplicity and/or integrated design of the expansion and locking assemblies disclosed herein, the prosthetic valves disclosed herein also can be cheaper and easier to manufacture.

Additional information and examples are provided below with reference to the accompanying drawings.

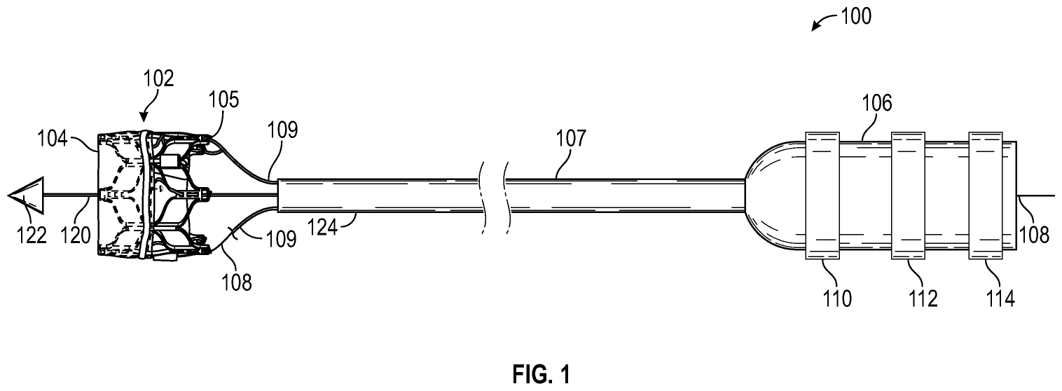

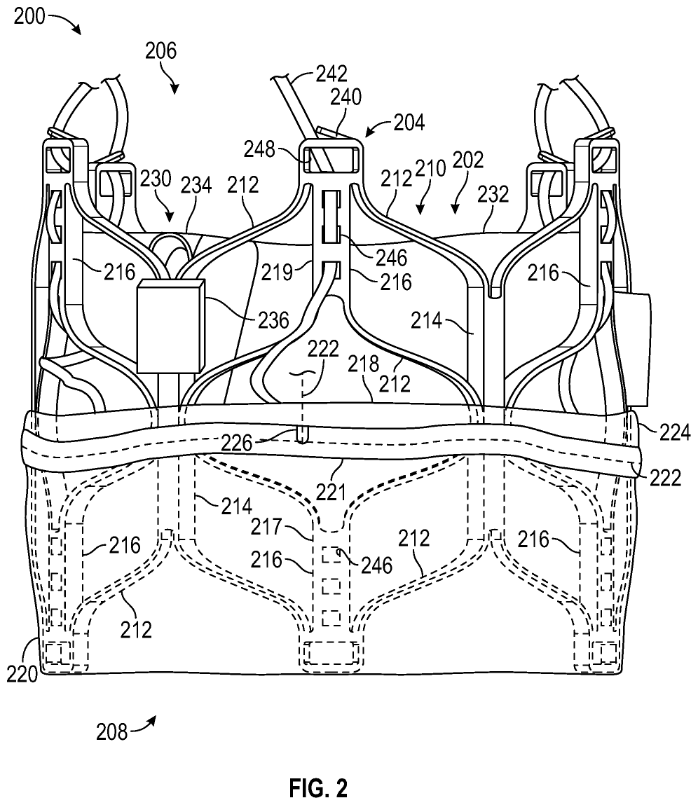

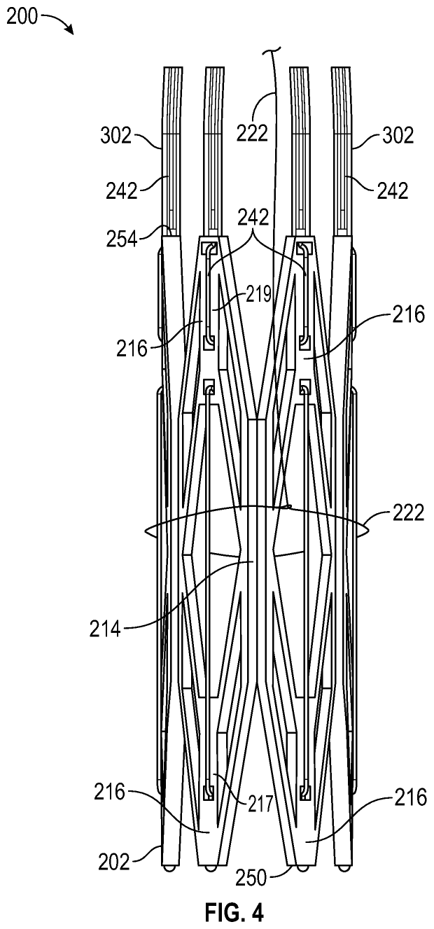

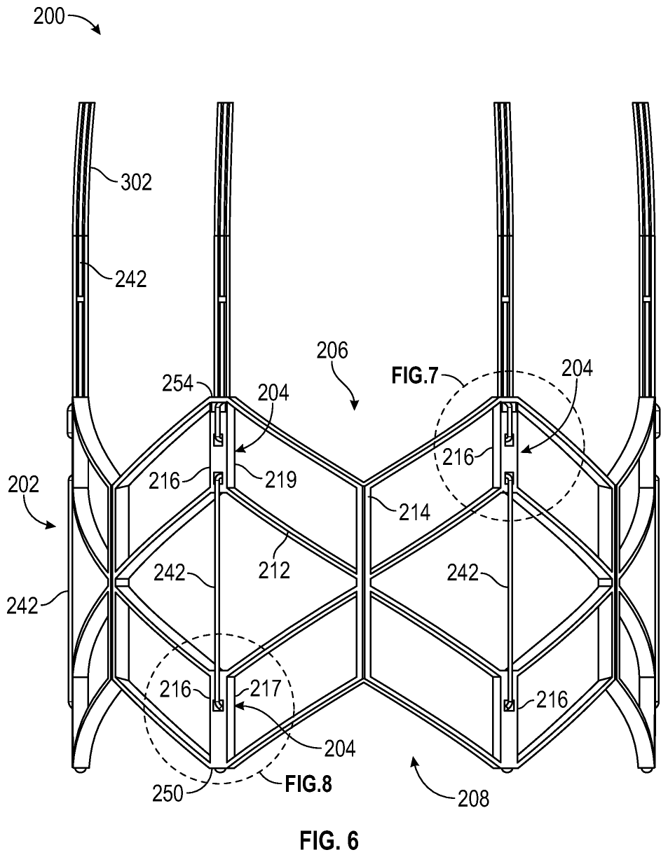

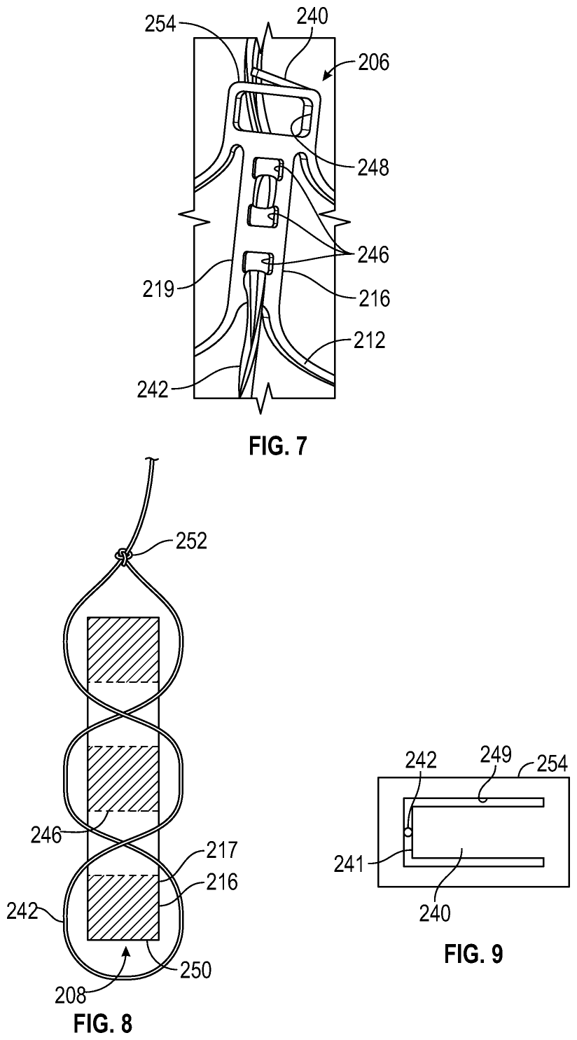

FIG. 1 shows an exemplary delivery assembly that can be used to deliver an exemplary prosthetic heart valve to a native heart valve. FIGS. 2-6 show an exemplary expansion and locking assembly that can be used to expand and/or lock an exemplary prosthetic heart valve. Specifically, FIGS. 2-3 and 6 show the exemplary prosthetic heart valve in a radially expanded state, FIG. 4 shows the exemplary prosthetic heart valve in a radially compressed state, and FIG. 5 shows the exemplary prosthetic heart valve in a partially radially expanded state (i.e., a state/position in-between the radially compressed state and the radially expanded state). FIG. 7 shows a close-up view of an exemplary locking mechanism that can be incorporated in a first portion of a frame of the exemplary prosthetic heart valve. FIG. 8 shows a close-up view of a second portion of the frame of the exemplary prosthetic heart valve that can incorporate a portion of the expansion and locking assembly, and FIG. 9 shows a top plan view of an outflow end of the second portion of the frame that can include a locking element of the expansion and locking assembly

FIG. 1 illustrates a delivery apparatus 100, according to one example, adapted to advance a prosthetic heart valve 102, such as prosthetic heart valve 200 described below in FIGS. 2-9, through a patient's vasculature and/or to deliver the prosthetic heart valve 102 to an implantation site (e.g., native heart valve) within a patient's body. The prosthetic heart valve 102 can be mounted on, retained within, and/or releasably coupled to a distal end portion of the delivery apparatus 100.

The prosthetic heart valve 102 can include a distal end 104 (which can be the inflow end of the prosthetic valve 102, such as when the prosthetic heart valve 102 is configured to replace a defective aortic valve when delivered transfemorally) and a proximal end 105 (which can be the outflow end of the prosthetic valve 102, such as when the prosthetic heart valve 102 is configured to replace a defective aortic valve when delivered transfemorally), wherein the proximal end 105 is positioned closer to a handle 106 of the delivery apparatus 100 than the distal end 104, and wherein the distal end 104 is positioned farther from the handle 106 than the proximal end 105. It should be understood that the delivery apparatus 100 and other delivery apparatuses disclosed herein can be used to implant prosthetic devices other than prosthetic valves, such as stents or grafts.

The delivery apparatus 100 in the illustrated example generally includes the handle 106, a first elongated shaft 107 (which comprises an outer shaft in the illustrated example) extending distally from the handle 106, one or more actuator members 108 extending distally through the shaft 107, and one or more support members 109 that can extend distally through the shaft 107 and can abut the proximal end 105 of the prosthetic valve 102. The delivery apparatus 100 can further include an inner shaft 120 extending from the handle 106 through the outer shaft 107 and a nose cone 122 connected to the distal end portion of the inner shaft 120.

Each actuator member 108 can have a distal end connected to the distal end of the prosthetic valve 102. Each of the actuator members 108 can extend through a respective support member 109 and together can define a respective actuator assembly that can extend through the shaft to the handle 106 (see also FIGS. 4-6). In alternative examples, the actuator members 108 and the support members 109 need not be co-axial with respect to each and instead can extend side-by-side through the shaft.

The actuator members 108 and/or the support members 109 can be configured to radially expand the prosthetic heart valve 102 by bringing the ends 104, 105 of the prosthetic valve 102 closer together (i.e., squeezing the prosthetic valve 102 axially) thereby axially foreshortening and radially expanding the prosthetic valve 102. As one example, the actuator members 108 can be configured to be actuated to provide a proximally directed (e.g., pulling) force to the distal end 104 of the prosthetic valve 102 while the one or more support members 109 can be configured to provide a countervailing distally directed (e.g., pushing) force to the proximal end 105 of the prosthetic valve 102. As one such example, a physician can pull the actuator members 108 to provide the proximally directed force to the distal end 104 of the prosthetic valve 102, while simultaneously gripping, holding, and/or pushing the handle 106 to provide the countervailing distally directed force to the proximal end 105 of the prosthetic valve 102.

As described in greater detail below, the actuator members 108 can cooperate with a locking element on the prosthetic valve 102 to retain the prosthetic valve in a radially expanded state.

The actuator members 108 can comprise a suture, string, cord, wire, cable, or other similar device that can transmit a pulling force from the handle 106 to the prosthetic valve when actuated by a physician. The support members 109 can comprise a relatively more rigid component, such a tube that can abut the proximal end 105 of the prosthetic valve 102 and resist proximal movement of the prosthetic valve relative to the shaft 107 when a proximal pulling force is applied to the actuator members 108.

Although two actuator members 108 and two support members 109 are shown in FIG. 1, it should be understood that the delivery apparatus 100 can include more or less than two actuator members 108 and/or two support members 109, in other examples. As just one example, the delivery apparatus 100 can include six actuator members 108 and/or six support members 109. In other examples, a greater or fewer number of actuator members 108 and/or support members 109 can be present, such as three, four, five, seven, and/or eight actuator members 108 and/or three, four, five, seven, and/or eight support members 109. In some examples, the delivery apparatus 100 can include equal numbers of actuator members 108 and support members 109. However, in other examples, the delivery apparatus 100 can include a different number of actuator members 108 than support members 109.

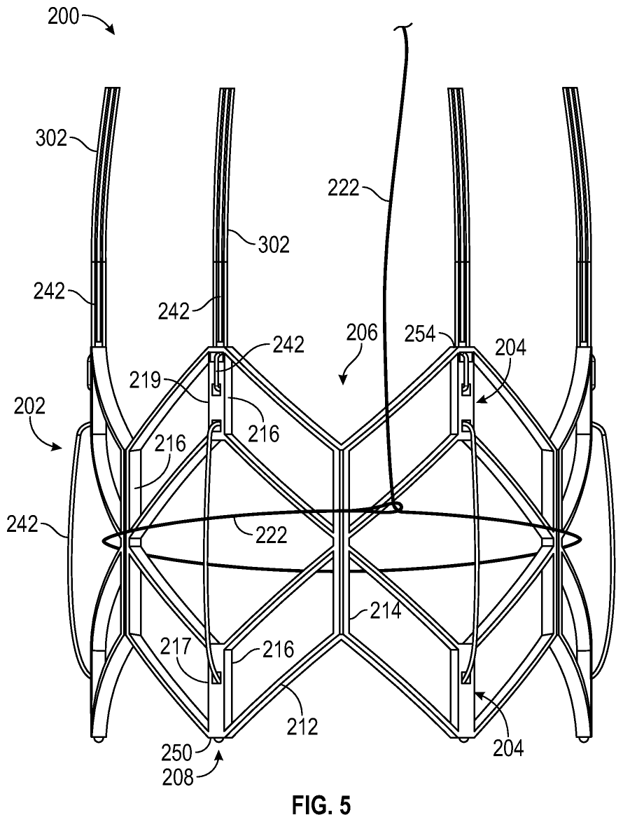

The prosthetic valve 102 can be at least partially self-expanding, in some examples. That is, the prosthetic valve 102 can be configured to self-expand from a radially compressed, delivery state to at least a partially radially expanded state. In one example, the prosthetic valve 102 can be configured to self-expand to a partially radially expanded state (e.g., FIG. 5), and the actuator members 108 can be actuated to further radially expand the prosthetic valve to the fully radially expanded state (e.g., FIG. 6). Thus, in such examples, the prosthetic valve 102 can be both mechanically expandable and self-expanding. As another such example, the prosthetic valve 102 can be configured to self-expand to the fully radially expanded state (e.g., FIG. 6) and the actuator members 108 can be actuated to only lock the prosthetic valve 102 in the fully radially expanded state. When configured to be self-expanding, the frame of the prosthetic valve 102 can be constructed from a shape-memory material (e.g., Nitinol) that biases the prosthetic valve 102 towards a radially expanded state. That is, the prosthetic valve 102 can be shape set in a partially radially expanded state or the fully radially expanded state (e.g., not the radially compressed state) so that the prosthetic valve 102 returns to the shape set radially expanded state when released from a restraining mechanism (e.g., lasso, sheath, etc.).

However, in other examples, the prosthetic valve 102 may not be self-expanding. For example, the frame of the prosthetic valve can be made from a plastically expandable material and can retain the prosthetic valve in its radially compressed state until expanded by the actuator members 108. In such examples, the actuator members 108 can be configured to expand the prosthetic valve all of the way from the crimped state to the fully radially expanded state.

In other examples, the frame of the prosthetic valve 102 can comprise a plurality of overlapping struts that are pivotably connected to each other at hinges where the struts overlap with each other, as further described in U.S. Publication Nos. 2018/0153689 and 2020/0188099, which are incorporated herein by reference. In some examples, the struts can be configured and connected to each other such that the prosthetic valve remains in a radially compressed state until expanded by actuation of the actuator members 108. In other examples, the struts can be configured and connected to each other such that the frame has some inherent resiliency that causes the frame to self-expand to at least a partially radially expanded state when released from a constraining member (e.g., a delivery sheath).

In some examples, the shaft 107 can have a distal end portion 124 sized to house the prosthetic valve in its radially compressed, delivery state during delivery of the prosthetic valve through the patient's vasculature. In this manner, the distal end portion 124 functions as a delivery sheath or capsule for the prosthetic valve during delivery, and as such may be referred to herein as delivery sheath 124.

The handle 106 of the delivery apparatus 100 can include one or more control mechanisms (e.g., knobs or other actuating mechanisms) for controlling different components of the delivery apparatus 100 in order to implant the prosthetic heart valve 102. For example, in the illustrated example the handle 106 can comprise one or more of first, second, and third knobs 110, 112, and 114.

When included, the first knob 110 can be configured to produce axial movement of the shaft 107 relative to the prosthetic heart valve 102 in the distal and/or proximal directions in order to deploy the prosthetic valve 102 from the delivery sheath 124 once the prosthetic valve 102 has been advanced to a location at or adjacent the desired implantation location within the patient's body. For example, actuating the first knob 110 in a first direction (e.g., clockwise) can retract the shaft 107 proximally relative to the prosthetic heart valve 102 and actuation of the first knob 110 in a second direction (e.g., counter-clockwise) can advance the shaft 107 distally. The first knob 110 can be actuated by rotating the knob 110 as indicated above, or by sliding or moving the knob 110 axially, such as by pulling and/or pushing the knob.

When included, the second knob 112 can be configured to actuate the actuator members 108 to radially expand and/or lock the prosthetic heart valve 102. For example, actuating the second knob 112 can pull the actuator members 108 proximally relative to the support members 109, thereby radially expanding the prosthetic heart valve 102 and locking the prosthetic heart valve 102 in its current state/position. The second knob 112 can be actuated by rotating the knob 112, or by sliding or moving the knob 112 axially, such as by pulling and/or pushing the knob.

In other examples, the second knob 112 may not be included, and a physician can expand and/or lock the prosthetic heart valve 102 by directly actuating (e.g., pulling) the actuator members 108. In such examples, the actuator members 108 can extend through and/or out of the handle 106 so that they are directly accessible to the physician and/or can be easily pulled by the physician. After radially expanding and locking the heart valve 102, the physician can cut the actuator members 108, such as at and/or near a locking element (e.g., locking element 240 described below) of the heart valve 102. The delivery apparatus 100 can include one or more cutting elements that can be actuated by the user to cut the actuator members 108 near the prosthetic valve, such as disclosed in U.S. Publication No. 2018/0153689.

When included, the third knob 114 can be configured to be actuated to retain the prosthetic heart valve 102 in its expanded configuration. For example, the third knob 114 can be operatively connected to a locking tool and can be actuated (e.g., rotated) to move the locking tool from a disengaged to an engaged state (to lock the prosthetic valve 102) and/or from the engaged state to the disengaged state (to unlock the prosthetic valve 102). For example, a physician can lock the prosthetic valve 102 to prevent the prosthetic valve 102 from collapsing and/or can unlock the prosthetic valve 102 to compress and/or reposition the prosthetic valve 102 relative to the native tissue. The third knob 114 can be actuated by rotating the knob 114, or by sliding or moving the third knob 114 axially, such as by pulling and/or pushing the knob.

However, in other examples, the third knob 114 may not be included. In some such examples, the prosthetic valve 102 can be self-locking and may not require any action from the physician to lock at a particular valve diameter. That is, the locking mechanism can automatically and/or continuously lock the prosthetic valve 102 at a range of valve diameters, without needing to be engaged/activated by the physician. For example, one such self-locking locking mechanism is shown in FIGS. 2, 3, and 7.

FIGS. 2-6 illustrate an exemplary example of a transcatheter prosthetic heart valve 200 that can be delivered to an implantation site (e.g., native heart valve) through a patient's vasculature (e.g., veins or arteries) using a delivery apparatus, such as the delivery apparatus 100 described above. The heart valve 200 includes a radially expandable and compressible annular frame 202 and an expansion and locking assembly 204 that is configured to radially expand the heart valve 200 and to lock the frame 202 at one or more valve diameters to prevent radial compression/collapse of the heart valve 200.

The heart valve 200 includes an outflow end 206 opposite an inflow end 208. When implanted in a native heart valve or vessel, blood is configured to flow from the inflow end 208 to the outflow end 206. In some examples, such as where the heart valve 200 is delivered to a native aortic valve through the aorta, the outflow end 206 can be a proximal end 206 of the prosthetic valve 200 (the end closer to the delivery apparatus) and the inflow end 208 can be a distal end 208 of the prosthetic valve 200 (the end furthest from the delivery apparatus). In other examples, such as where the heart valve is delivered to a native mitral valve through the atrial septum and right atrium or to the native aortic valve through the apex of the heart, the outflow end 206 can be a distal end 206 of the prosthetic valve 200 and the inflow end 208 can be a proximal end 208 of the prosthetic valve 200.

The heart valve 200 is radially compressible and expandable between a radially compressed state (FIG. 4) and a radially expanded state (FIGS. 2-3, and 6). The heart valve 200 can be advanced through a patient's vasculature in the radially compressed state and can then be radially expanded at the implantation site (e.g., aortic and/or mitral valve) to the radially expanded state. The radially expanded state can be a fully radially expanded state where the heart valve 200 is configured to circumferentially contact and/or create a seal with the surrounding native tissue (when implanted at the implantation site) such that blood only flows through the heart valve 200 and does not flow around the heart valve 200, between the heart valve 200 and the native tissue.

The heart valve 200 also can be configured to be radially expanded to, and locked in, one or more partially radially expanded states (e.g., FIG. 5), between the radially compressed state and the radially expanded state. As just one example, such as where the prosthetic heart valve 200 is configured to be partially self-expanding, the prosthetic heart valve 200 can be configured to self-expand to a first partially radially expanded state and can then be mechanically expanded to, and continuously locked in, a plurality of partially radially expanded states between the first partially radially expanded state and the radially expanded state. As another example, such as where the heart valve 200 is not self-expanding, the heart valve 200 can be configured to be mechanically expanded and continuously locked in a plurality of a partially radially expanded states between the radially compressed state and the radially expanded state.

The frame 202 of the heart valve 200 can include a plurality of struts 210 defining a plurality of windows or openings. The struts 210 can include rows of angled struts 212 that extend between a first set of vertical struts or posts 214 and a second set of vertical struts or posts 216. The first and second sets of vertical struts 214, 216 can extend axially between the outflow end 206 and the inflow end 208 of the prosthetic valve 200. The first set of vertical struts or posts 214 can be configured to at least partially form, support, and/or define one or more commissures of the leaflet assembly of the prosthetic valve 200. The second set of vertical struts or posts 216 can be configured to at least partially form, support, and/or define the expansion and locking assembly 204. In this way, the expansion and locking assembly 204 can be at least partially integrated/incorporated into the frame 202 of the prosthetic valve 200, thereby reducing the diameter and/or profile of the frame 202 in the radially compressed (i.e., crimped) state.

The second set of vertical struts 216 can comprise two separate rows of struts that are axially spaced from one another, namely a first row of vertical struts 217 at the inflow end 208 of the frame 202, and a second row of vertical struts 219 at the outflow end 206 of the frame 202. As will be explained in greater detail below, the expansion and locking assembly 204 can extend axially between these two rows of vertical struts 217, 219 to pull the inflow and outflow ends 208, 206 of the frame 202 towards one another to radially expand the prosthetic valve 200. Because the first row of vertical struts 217 form and/or define the inflow end portion of the frame, the first row of vertical struts 217 also can be referred to herein as the inflow end portion 217 of the frame 202. Similarly, the second row of vertical struts 219 also can be referred to herein as the outflow end portion 219 of the frame 202.

The frame 202 can be made of any of various suitable plastically-expandable materials (e.g., stainless steel, etc.) or self-expanding materials (e.g., nickel titanium alloy (NiTi), such as nitinol) as known in the art. When constructed of a plastically-expandable material, the frame 202 (and thus the prosthetic heart valve 200) can be crimped to the radially compressed state on a delivery catheter and then expanded inside a patient by, for example, the expansion and locking assembly 204. When constructed of a self-expandable material, the frame 202 (and thus the prosthetic heart valve 200) can be crimped to the radially compressed state and restrained in the radially compressed state by a lasso, delivery sheath, and/or other restraining mechanism. Once inside the body, the restraining force of the restraining mechanism can be released (e.g., reducing tension in a lasso or deploying the prosthetic valve from a sheath), which allows the prosthetic valve 200 to radially expand.

Suitable plastically-expandable materials that can be used to form the frame 202 include, without limitation, stainless steel, a biocompatible, high-strength alloy (e.g., a cobalt-chromium or a nickel-cobalt-chromium alloys), polymers, or combinations thereof. In particular examples, frame 202 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

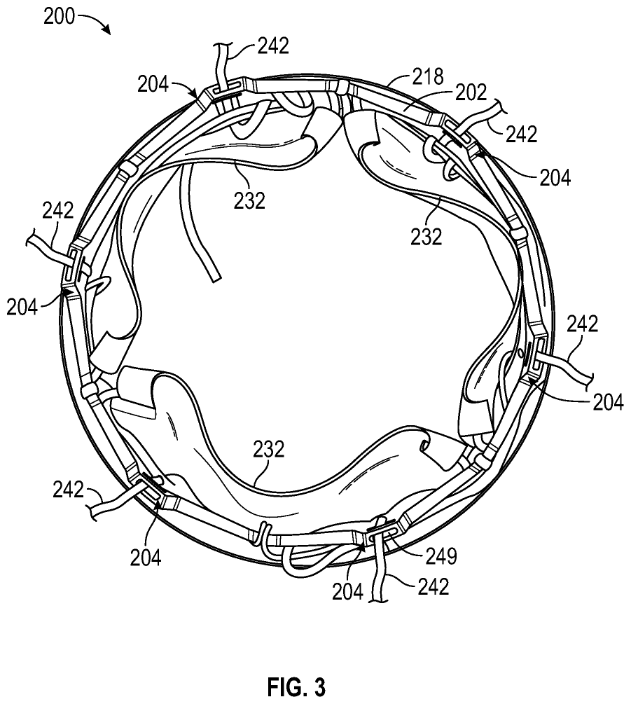

The prosthetic valve 200 can include a skirt assembly 218 (FIGS. 2-3) that is configured to cover the openings of the frame 202 and provide a seal between the frame 202 and the surrounding native tissue. The skirt assembly 218 can extend from and/or around the inflow end 208 of the prosthetic valve 200 along the inner and/or outer sides of the frame 202 (towards the outflow end 206 of the prosthetic valve 200). The skirt assembly 218 can include an outer skirt 220 that extends circumferentially around the outer surface of the frame.

The outer skirt 220 can extend from the inflow end 208 of the prosthetic valve 200 along the outer surface of the frame and can be secured to the frame 202, such as via sutures. For example, the outer skirt 220 can be sutured to the angled struts 212, the vertical struts 214, and/or to an inner skirt (not shown) that is positioned on the opposite surface of the frame. In some examples, the outer skirt 220 can extend from the inflow end 208 towards the outflow end 206 but can stop short of the flexible connectors (e.g., flexible connectors 236 described below) that hold the commissures of the leaflets of the prosthetic valve to the frame.

In some examples, the skirt assembly 218 can include an inner skirt (not shown) that can extend from the inflow end 208 of the prosthetic valve 200 along the inner surface of the frame 202 and can be secured to the frame 202, such as via sutures. For example, the inner skirt can be sutured to the angled struts 212, the vertical struts 214, and/or can be sutured to the outer skirt 220.

The skirt assembly 218 can be formed of and/or constructed from one or more materials that are flexible enough to allow for radial compression and expansion of the frame 202 and/or that are tough enough to resist tearing and prevent paravalvular leakage (PVL). As examples, the skirt assembly 218 can be formed of and/or constructed from one or more of foam, cloth, fabric, pericardial tissue, and/or polymers, such as such as polyethylene terephthalate (PET) and/or an expanded polytetrafluoroethylene (ePTFE). In some examples, the skirt assembly 218 can be configured to promote tissue ingrowth to further enhance the seal between the prosthetic heart valve 200 and the native heart valve tissue. For example, the portion of the skirt assembly 218 included on the outer side of the frame 202 (e.g., the outer skirt) can includes pores and/or can be impregnated with growth factors such as transforming growth factor alpha (TGF-alpha) to promote tissue ingrowth.

The outer skirt 220 can include a sleeve 221 (FIG. 2) that is configured to receive a restraining mechanism (e.g., a tension member such as in the form of a lasso or adjustable loop) 222, such as in examples where the prosthetic valve 200 is self-expanding and requires a restraining mechanism 222 (FIGS. 2 and 4-5) to hold the prosthetic valve 200 in the radially compressed state and/or to control expansion of the prosthetic valve. As one example, the sleeve 221 can form a pocket in the outer skirt 220 that extends circumferentially around the frame 202. The sleeve 221 can be included at an outflow edge portion 224 of the outer skirt. The sleeve 221 can be formed by folding the outflow edge portion back against the main body of the skirt and then connecting (e.g., stitching) the folded flap against the main body of the skirt. The sleeve 221 desirably is positioned approximately halfway between the inflow end 208 and the outflow end 206 of the prosthetic valve 200 so that the restraining force of the retraining mechanism is equally distributed along the length of the frame.

The restraining mechanism 222 can extend through the sleeve 221 and circumferentially around the frame 202. The restraining mechanism 222 can exit the sleeve 221 via an opening 226 in the skirt assembly 218. From the opening 226, the restraining mechanism 222 can extend proximally towards the handle of the delivery apparatus and can be configured to be adjusted by the physician to allow the prosthetic valve 200 to be radially expanded. For example, the physician can loosen the restraining mechanism 222 to allow the prosthetic valve 200 to radially self-expand and/or to be mechanically radially expanded. The restraining mechanism 222 can be adjusted between a constrained state in which the restraining mechanism 222 is configured to hold the prosthetic valve 200 in the radially compressed state and a loosened state in which the restraining mechanism 222 is configured to allow the prosthetic valve 200 to radially expand.

The prosthetic valve 200 can include a leaflet assembly 230 (FIGS. 2-3) comprising leaflets 232. The leaflets 232 are configured to selectively open and close to regulate the flow of blood through the prosthetic valve 200. In the illustrated example (FIG. 3), the leaflet assembly 230 includes three flexible leaflets 232, although a greater or a smaller number of leaflets can be used. The leaflets 232 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein. Additional information regarding the leaflets 232, as well as additional information regarding the material of the skirt assembly 218, can be found, for example, in U.S. Pat. No. 10,195,025, which is incorporated herein by reference.

The leaflets 232 can be secured to one another at their adjacent sides to form commissures 234. The prosthetic valve 200 can include flexible connectors 236 (FIG. 2) that can be used to mount the leaflets 232 to the vertical struts 214 at the commissures 234 and/or to interconnect pairs of adjacent sides of the leaflets 232. For example, the prosthetic valve 200 can include three flexible connectors 236 to secure each of the three leaflets 232 to the frame 202 at the three commissures 234. The flexible connectors 236 can be mounted to the vertical struts 214 more proximate the outflow end 206 than the inflow end 208, between adjacent rows of the angled struts 212. The inflow edge portions of the leaflets 232 can be secured to the frame 202, such by stitching the inflow edge portions leaflets directly to selected struts of the frame, or by stitching the inflow edge portions of the leaflets to an inner skirt, which in turn is connected to selected struts of the frame with sutures. Further details regarding the formation of the commissures and connecting the commissures and the inflow edge portions of the leaflets are disclosed in U.S. Patent Application Nos. 63/085,947, 63/139,514, 63/026,866, and 63/224,534, which are incorporated herein by reference.

As introduced above, the expansion and locking assembly 204 is configured to radially expand the prosthetic valve 200, such as after the restraining mechanism 222 has been loosened, and to lock/hold the prosthetic valve 200 in a radially expanded state. For example, in operation, a physician can first loosen the restraining mechanism 222 (in examples where the prosthetic valve 200 is self-expanding), and can then adjust the expansion and locking assembly 204 to further radially expand the prosthetic valve 200 and to lock the prosthetic valve 200 in a radially expanded state.

The expansion and locking assembly 204 can comprise a locking element 240 and an actuator member 242. The actuator member 242 is coupled to the frame at first location (which can be at or closer to the inflow end of the frame) and at a second location by the locking element (which can be at or closer to the outflow end of the frame), wherein the second location is axially spaced from the first location. In particular, the actuator member is fixedly secured to the frame at the first location and slidably coupled to the locking element 240 at the second location such that actuator member can be pulled in a proximal direction relative to the locking. The locking element 240 is configured to hold/lock the prosthetic valve 200 in a radially expanded state to prevent radial compression of the frame 202 by preventing movement of the actuator member 242 in the distal direction relative to the locking element. The actuator member 242 is configured to be selectively adjusted (e.g., pulled) to move the proximal direction to cause radial expansion of the frame 202 or to simply the lock the frame in the radially expanded state if the frame is fully self-expandable. For example, a physician can pull the actuator member 242 proximally to radially expand the prosthetic valve 200.

The actuator member 242 desirably comprises a flexible tension member, such as a suture (such as a Dyneema® suture), string, cord, wire, cable, or other similar device that can be readily pulled by a physician to radially expand the prosthetic valve 200 and can be the same as, or similar to, actuator members 108 described above with reference to FIG. 1. As illustrated in FIG. 3, the prosthetic valve 200 can include six actuator members 242. However, in other examples, the prosthetic valve 200 can include more or less than six actuator members 242.

The actuator members 242 can be coupled to the frame 202, and more specifically, can be coupled the frame 202 at or near the inflow and outflow ends 208, 206 of the frame, such that pulling of the actuator members 242 in the proximal direction while applying a distally directed force to the frame (e.g., via support members 109 or 302) causes the frame 202 to axially foreshorten and radially expand. As one example, the actuator members 242 can extend through openings 246 and/or 248 in the frame 202 at or near the inflow and outflow ends 208, 206 of the prosthetic valve 200. The openings 246, 248 can extend radially all of the way through the frame (from the inner side of the frame 202 to the outer side of the frame 202). In some examples, the openings 246 and/or 248 can be formed in the frame 202 by, for example, laser cutting. However, in other examples, the openings 246 and/or 248 can be formed by other removal means, such as die cutting, machining, etc., and/or can be integrally formed in the frame 202 during formation of the frame, such as via a molding process.

The openings 246, 248 can be included in the vertical struts 216 of the frame 202 and can be axially spaced from one another. However, in other examples, the actuator member 242 need not extend through the openings in the frame 202 and instead can extend next to the frame 202 and can be coupled to the vertical struts 216 via fasteners, adhesives, welding, and/or other fastening means. Each of the actuator members 242 can be coupled to, and extend between, one of the vertical struts in the first row of vertical struts 217 and one of the vertical struts in the second row of vertical struts 219 that are circumferentially aligned with one another. The actuator member 242 can extend axially between the vertical struts 217 on the inside of the outer skirt, such between the outer skirt and the outer surface of the frame, or along the inner surface of the frame, and/or on the outside of the outer skirt.

For example, as illustrated in FIGS. 6-8, each of the actuator members 242 can extend axially along, and be coupled to, one of the vertical struts 217 at the inflow end 208 of the frame 202 and the circumferentially aligned vertical strut 219 at the outflow end 206 of the frame 202. In the example illustrated in FIG. 8, the actuator member 242 can be wrapped around an inflow end or apex 250 of the vertical struts 217 and can be looped back through the openings 246 of the vertical strut 216 in a crisscross manner. A knot 252 can be formed in each actuator member 242 at the end of the vertical strut 217 opposite the inflow apex 250 to couple the actuator member 242 to the vertical strut 217. However, in other examples, each of the actuator members 242 can be tied in a knot at the inflow apex 250 and may only weave in and out of the openings 246 in an alternating manner and may not double back through the openings 246 in a crisscross manner. In yet further examples, the actuator members 242 may not extend through any openings of the vertical struts 217 and instead only may be secured to the vertical struts 217, such as at the inflow apex 250. As discussed above, the actuator members 242 can be tied in a knot to secure the actuator members 242 to the vertical struts 217. However, in other examples, other fastening means such as sutures, fasteners, adhesives, welding, etc., can be used to secure the actuator members 242 to the vertical struts 217.

In the example illustrated in FIG. 8, the vertical struts 217 at the inflow end 208 of the frame 202 include two of the openings 246 through which the actuator members 242 extend. However, in other examples, the vertical struts 217 can include more or less than two openings 246 through which the actuator members 242 extend. For example, the vertical struts 217 can include only one opening 246 (FIGS. 4-6). As another example, the vertical struts 217 can include three openings 246 (FIGS. 2-3).

The actuator members 242 can extend axially from the first row of vertical struts 217 at the inflow end 208 of the frame 202 to the second row of vertical struts 219 at the outflow end 206 of the frame 202. As introduced above, the actuator members 242 can extend axially between the vertical struts 217 on the inside of the skirt assembly 218, such as radially inward from the skirt assembly 218 (e.g., within the lumen of the prosthetic valve 200), within the skirt assembly 218 itself (e.g., between the inner and outer skirts of the skirt assembly 218), and/or on the outside of the skirt assembly 218, such as radially outward from the skirt assembly 218.

In the example illustrated in FIG. 7, the actuator members 242 can be configured to weave in and out of the openings 246 of the vertical struts 219 at the outflow end 206 of the prosthetic valve 200 in an alternating manner. For example, the actuator members 242 can be configured to extend in a first direction through a first one of the openings 246 in the second row of vertical struts 219 positioned farthest from the outflow end 206, and then in an opposite second direction through an adjacent second one of the openings 246, and then back through a third one of the openings 246 in the first direction (FIGS. 2, 7). The actuator members 242 can be configured to weave back through the opening 248, such as in the second direction, and can then extend through axially extending openings 249 (FIGS. 3 and 9) in the apices 254 of the vertical struts 219.

However, in other examples, the vertical struts 219 can include more or less than three openings 246 through which the actuator members 242 extend. For example, the vertical struts 219 can include only two openings 246 (FIGS. 4-6) through which the actuator members 242 extend. The actuator members 242 frictionally engage the vertical struts 219 in the regions of the vertical struts 219 where the actuator members 242 extend through the vertical struts 219 (e.g., at or near the openings 246, 248). This frictional engagement between the vertical struts 219 and the actuator members 242 can provide further holding force (in addition to holding force provided by the locking elements 240) that can help prevent the actuator members 242 from sliding distally. Thus, weaving the actuator members 242 through the opening 246, 248 in the vertical struts 219 can help prevent radial compression of the prosthetic valve by frictionally resisting sliding of the actuator members 242 in the distal direction relative to the vertical struts 219.

In yet further examples, the actuator members 242 may not extend through any openings of the vertical struts 219 and instead only may be secured to the vertical struts 219, via a knot, fastener, adhesives, welding, and/or other suitable fastening means.

The actuator members 242 can extend proximally from the proximal apex 254 of the struts 219 and the locking element 240 towards, to, and/or out of the handle of the delivery apparatus, as discussed above with reference to FIG. 1. In some examples (FIGS. 4-6), the actuator members 242 can extend through support members 302 (e.g., through a lumen of the support members 302) of the delivery apparatus (e.g., delivery apparatus 100). Support members 302 may be the same as, or similar to, support members 109 described above with reference to FIG. 1. The support members 302 can abut the proximal apex 254 of the vertical struts 219 to provide a distally directed force to the outflow end 206 of the frame 202 that helps radially expand the prosthetic valve 200 and/or that keeps the prosthetic valve 200 in place relative to the surrounding native tissue during the radial expansion of the prosthetic valve 200, as discussed above with reference to FIG. 1.

The opening 248 can be configured to have a wider and/or larger cross-section than the openings 246, in some examples. The larger cross-section of the opening 248 can allow a physician to move the actuator members 242 into and out of engagement with the locking element 240. As one example, the physician can slide the actuator members 242 laterally into and out of engagement with the locking element 240, such as in examples where the locking element 240 comprises a spring tooth or the like.

Each locking element 240 can comprise a pivoting or deformable spring tooth that is biased towards a proximal apex 254 of a vertical struts 219 (such that the locking element 240 lies substantially flat on and/or flush with the proximal apex 254 the vertical strut 219) to apply pressure to the actuator member 242 extending through openings 249 to prevent the actuator member 242 from sliding distally through the opening 249. That said, the locking element 240 can pivot away from the proximal apex 254 of the vertical strut 219 to accommodate movement (e.g., sliding) of the actuator member 242 in the proximal direction, such as when radially expanding the prosthetic valve 200. The locking element 240 may provide relatively little resistive force (e.g., approximately 1.5 N) to proximal movement of the actuator member 242, thereby allowing a physician to pull the actuator members 242 with relatively little effort/force. Weaving the actuator members 242 through the openings 246 and/or 248 in the vertical struts 217 and/or 219 also can provide additional frictional resistance to undesirable distal movement of the actuator members 242. In this way, weaving the actuator members 242 through the openings 246 and/or 248 can help prevent radial collapse of the prosthetic valve.

As best shown in FIG. 9, the locking elements 240 can be integrally formed in the vertical struts, such as by laser cutting the apices 254 to form the openings 249 and the locking elements 240, which are connected at one end to the apices 254. The opening 249 can be U-shaped as shown. In other examples, the locking elements 240 can be separately formed and subsequently attached to the apices 254 and/or contained within the openings 249.

The locking element 240 has a free edge 241 that can capture and frictionally engage an actuator member 242 between the free edge 241 and an opposing surface of the opening 249. In its natural (non-deformed) state, the locking element 240 can reside entirely within the opening 249. Threading or inserting an actuator member 242 through the opening 249 can causes the locking element 240 to deform slightly and bend away from the apex 254 (see FIG. 7). The locking element 240 is biased toward the apex 254 so as to maintain contact with the actuator member 242.

By utilizing a relatively thin and flexible tension member, such as a thin cord, wire, string, suture, or the like for the actuator members 242 and/or by least partially incorporating/integrating the expansion and locking assembly 204 into the frame 202 of the prosthetic heart valve 200 as described above (e.g., by weaving the actuator members 242 through the openings 246, 248 in the frame 202 and/or by including the locking elements 240 at the proximal apex 254 of the vertical struts 219), the overall size of the expansion and locking assembly 204 can be significantly reduced compared to other mechanical expansion and locking assemblies. The slimmer expansion and locking assembly 204 disclosed herein can reduce the overall size of the prosthetic valve 200, such that the prosthetic valve 200 has a smaller diameter and/or slimmer profile in the radially compressed state. This can minimize risks and/or complications for the patient as the prosthetic valve 200 is advanced through the patient's vasculature. Further, because the actuator members 242, locking elements 240, and openings 246, 248 are simpler, less expensive, and/or easier to manufacture than known expansion and locking assemblies, the prosthetic valve 200 itself can be simpler, less expensive, and/or easier to manufacture.

In operation, the prosthetic valve 200 can be loaded/mounted onto the delivery apparatus (e.g., delivery apparatus 100) in the radially compressed (crimped) state. In some examples, the restraining mechanism 222 (e.g., lasso) can be used to hold the prosthetic valve 200 in the radially compressed state. The prosthetic valve 200 optionally can be loaded inside a delivery sheath (e.g., sheath 124) of the delivery apparatus. The prosthetic valve 200 can then be advanced through the patient's vasculature by the delivery apparatus until it reaches the implantation site (e.g., native aortic valve). As one example, the prosthetic heart valve 200 can be delivered to the native aortic valve, such as transfemorally. For example, the prosthetic heart valve 200 can be introduced into a femoral artery through an incision in a patient's groin and can then be advanced through the femoral artery to the aortic valve via the iliac artery and aorta. As another example, the prosthetic heart valve 200 can be introduced to the native mitral valve, such as transfemorally. For example, the prosthetic heart valve 200 can be introduced into a femoral vein through an incision in a patient's groin and can then be advanced through the femoral vein to the mitral valve via the inferior vena cava, the right atrium, and an opening in the atrial septum. In other examples, the prosthetic heart valve 200 can be implanted in various other locations, such as in the native tricuspid valve, within the superior vena cava or inferior vena cava (for replacing the function of the native tricuspid valve), within the native pulmonary valve, or within the pulmonary artery (for replacing the function of the native pulmonary valve).

Use of a delivery sheath to restrain the prosthetic valve (in addition the restraining mechanism) during delivery can be advantageous in that the delivery sheath can provide an atraumatic covering for the prosthetic valve as it is advanced through the patient's vasculature. Once deployed from the delivery sheath at or near the implantation site, the restraining mechanism continues to hold the prosthetic valve in the radially compressed state during final positioning of the prosthetic valve. In other examples, the restraining mechanism can be removed, and the prosthetic valve can be retained in a radially compressed state only with the delivery sheath.

Upon reaching the implantation site, the physician can begin the deployment process, which can include deploying the prosthetic valve from the sheath (if contained within a sheath) and then releasing the prosthetic valve from the restraining mechanism 222, radially expanding the prosthetic valve 200 to the fully radially expanded state (FIG. 6) and locking the prosthetic valve 200 in the fully radially expanded state. In examples where the restraining mechanism 222 is included, such as in examples where the prosthetic valve 200 is at least partially self-expanding, the physician can start the deployment process by loosening the restraining mechanism 222, allowing the prosthetic valve to radially expand to a partially expanded state (FIG. 5). In the partially expanded state (FIG. 5) there may be some slack in the actuator members 242. Specifically, as the prosthetic valve 200 self-expands, the inflow and outflow ends 208, 206 of the frame 202 may be drawn towards one another, thereby creating slack in the actuator members 242 due to the fact that the actuators 242 are secured to the struts 217 at the inflow end portion of the prosthetic valve 200 and are held by the locking elements 240 at the outflow end portion of the prosthetic valve 200. In other words, during the initial expansion of the prosthetic valve, the actuators 242 are not pulled or moved proximally through the locking elements 240 to take up slack in the actuator members as the prosthetic valve expands. Thus, in the radially compressed state (FIG. 4) the actuator members 242 may be in a taut state, and as the prosthetic valve 200 radially self-expands and axially foreshortens, the actuator members 242 may relax to a loosened state (FIG. 5) in which slack exists in the actuator members 242.

If needed, the prosthetic valve can be recompressed to a smaller diameter or to the fully compressed state (FIG. 4) by actuating the restraining member 222 for re-positioning the prosthetic valve or for withdrawing the prosthetic out of the patient (in which case, the prosthetic valve can be first withdrawn back into the delivery sheath and then withdrawn from the patient's body). Advantageously, due to the slack in the actuator members 242 in the partially expanded state, the prosthetic valve can be recompressed to a smaller diameter or to the fully compressed state (FIG. 4) without having to manipulate or unlock the actuator members 242 from the locking elements 240.

After the prosthetic valve has been positioned at the desired implantation location and expanded to the partially expanded state, the physician may begin to mechanically radially expand the prosthetic valve by, for example, pulling the actuator members 242 proximally and/or by providing a countervailing distally directed pushing force to the outflow end 206 of the prosthetic valve 200 via, for example, the support members 302. As the physician pulls the actuator members 242 proximally relative to the frame 202 and the support members 302, the inflow and outflow ends 208, 206 of the frame 202 may be drawn towards one another as the prosthetic valve 200 radially expands. The proximally directed force provided by the physician initially removes the slack from the actuator members 242 and draws the actuator members 242 taut, which locks the prosthetic valve 200 (i.e., prevent radial re-compression of the prosthetic valve 200). Then, once the actuator members 242 are drawn taut, the physician can continue pulling the actuator members 242 proximally to mechanically radially expand the prosthetic valve 200.

In other examples, the prosthetic valve 200 may self-expand to the fully radially expanded state (FIG. 6) and may not need to be mechanically radially expanded. In such examples, the expansion and locking assembly 204 serves only as a locking assembly lock the prosthetic valve 200 in the fully expanded state. Moreover, in such examples, the actuator members 242 can form slack between the inflow and outflow ends of the prosthetic valve when the prosthetic valve is in the fully expanded state in order to facilitate recompression of the prosthetic valve, as described above. Thus, after the prosthetic valve 200 has self-expanded to the radially expanded state, the physician can pull the actuator members 242 proximally to remove the slack in the actuator members 242 and lock the prosthetic valve 200.

That is, in both of the above examples, because the actuator members 242 may be continuously engaged by the locking elements 240, once the physician removes the slack in the actuator members 242 and draws the actuator members 242 taut, the locking elements 240 can prevent the prosthetic valve from radially compressing, thereby locking the prosthetic valve 200 in the radially expanded state.

In some examples, the actuator members 242 can always be engaged with (and thus be prevented from moving distally by) the locking elements 240, such as during the entire deployment process and/or before the deployment, such as when the prosthetic valve 200 is loaded onto the delivery apparatus. In other examples, a physician can selectively engage the actuator members 242 with the locking element 240 when locking of the prosthetic valve 200 is desired (e.g., by sliding the actuator members 242 under the locking elements 240 as described above). In either of the above examples, whenever the actuator members 242 are engaged with the locking elements 240, the locking elements 240 are configured to provide a continuous locking force to the actuator members 242 to hold/lock the prosthetic valve 200 (i.e., prevent radial compression of the prosthetic valve 200) at any valve diameter. In this way, the locking elements 240 can continuously lock the prosthetic valve 200 such that, in examples where the prosthetic valve 200 is partially self-expanding, the locking elements 240 can continuously lock the prosthetic valve 200 at any state between the partially radially expanded state (FIG. 5) and the fully radially expanded state (FIG. 6).

In other examples, the prosthetic valve 200 can be plastically expandable and the restraining member 222 need not be used. Instead, once positioned at the desired implantation site, the prosthetic valve can be expanded by actuating the actuator members 242 to expand the prosthetic valve from the compressed, delivery state to the expanded state. During expansion, the frame undergoes plastic deformation and can generally retain the prosthetic valve in its radially expanded state. However, it has been found that even plastically expandable frames can experience a small amount of recoil that causes the prosthetic valve to slightly reduce in diameter after the expansion forces applied to the prosthetic valve are removed. The locking elements 240 advantageously lock the prosthetic valve in the expanded state to prevent or minimize the effects of frame recoil.

Once the actuator members 242 are drawn taut and are engaged with the locking elements 240, the prosthetic valve 200 cannot be radially re-compressed, unless the physician selectively disengages the actuator members 242 from the locking elements 240 and tightens the restraining mechanism 222.

Because the physician can pull the actuator members 242 without having to worry about the prosthetic valve 200 collapsing (because the locking elements 240 can continuously lock the actuator members 242), the expansion of the prosthetic valve 200 can be relatively tightly controlled/regulated. For example, the physician can adjust the rate of expansion based on how hard or far they pull the actuator members 242. The physician also can periodically pause and/or reverse expansion by ceasing to pull on the actuator members 242 and/or by tightening the restraining mechanism 222. Moreover, the physician can more easily radially expand and lock the prosthetic valve (i.e., by simply pulling the actuator members 242) as compared to other expansion and locking mechanisms, such as mechanisms that require a physician to rotationally thread a locker.

After locking the prosthetic valve 200 in the radially expanded state, a physician can cut and/or otherwise sever the actuator members 242, such as at and/or near the locking element 240 and withdraw and/or remove the delivery assembly (e.g., delivery apparatus 100), including the support members 302, from the patient.

As noted above, prior to the actuator members 242 being pulled taut to lock the prosthetic valve in its expanded state, the prosthetic valve can be radially re-compressed by simply tightening the restraining mechanism 222, even if the actuator members 242 are engaged with the locking elements 240. This is because, as described above, the actuator members 242 can be in a loose state (i.e., there can be slack in the actuator members 242) after the prosthetic valve 200 self-expands to the partially radially expanded state and/or the fully radially expanded state. This slack in the actuator members 242 allows the prosthetic valve 200 to be radially re-compressed without having to disengage the actuator members 242 from the locking elements 240. Thus, a physician can easily radially re-compress the prosthetic valves 200 (such as to reposition the prosthetic valve 200) without having to release/disengage the actuator members 242 from the locking elements 240.

Moreover, the slack in the actuator members 242 can facilitate the assembly process for the prosthetic valve. During manufacture, the frame of the prosthetic valve may need to be radially compressed and expanded any number of times, such as when assembling components to the frame. The slack in the actuator members easily allows the assembler to compress the frame of the prosthetic heart valve without having to unlock or otherwise manipulate the actuator members to permit radial compression of the frame.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve, comprising:
an annular frame comprising an inflow end and an outflow end, wherein the frame is radially compressible and expandable between a radially compressed state and a radially expanded state, and wherein the frame is configured to radially self-expand from the radially compressed state to at least a partially radially expanded state;
an actuator member coupled to the frame at first and second axially spaced locations, wherein the actuator member is configured to apply a proximally directed force to the frame to radially expand and/or lock the prosthetic heart valve; and
a locking element coupled to the frame at the second location, wherein the locking element is configured to allow the actuator member to slide in only a proximal direction relative to the frame and to continuously engage the actuator member to prevent the actuator member from sliding in an opposite distal direction past the locking element.

Example 2. The prosthetic heart valve of any example herein, particularly example 1, wherein the prosthetic heart valve is fully self-expanding and is configured to radially self-expand to the radially expanded state when released from a restraining mechanism.

Example 3. The prosthetic heart valve of any example herein, particularly example 1, wherein the prosthetic heart valve is partially self-expanding and is configured to radially self-expand to a partially radially expanded state when released from a restraining mechanism, wherein the partially radially expanded state is a state between the radially compressed state and the radially expanded state.

Example 4. The prosthetic heart valve of any example herein, particularly any one of examples 2 or 3, wherein the locking element is configured to continuously engage the actuator member while the prosthetic heart valve radially self-expands such that slack develops in the actuator member between the first and second locations as the valve axially foreshortens during the radial expansion.

Example 5. The prosthetic heart valve of any example herein, particularly any one of examples 3 or 4, wherein the actuator member is configured to radially expand the valve from the partially radially expanded state to the radially expanded state.

Example 6. The prosthetic heart valve of any example herein, particularly any one of examples 1-5, wherein the locking element is integrally formed in the frame.

Example 7. The prosthetic heart valve of any example herein, particularly any one of examples 1-6, wherein the frame comprises vertical struts at the first and second locations of the frame, and wherein the actuator member extends through one or more openings in one or more of the vertical struts.

Example 8. The prosthetic heart valve of any example herein, particularly example 7, wherein the actuator member weaves in and out of the openings in one or more of the vertical struts.

Example 9. The prosthetic heart valve of any example herein, particularly any one of examples 7 or claim 8, wherein the actuator member crisscrosses through the openings in one or more of the vertical struts.

Example 10. The prosthetic heart valve of any example herein, particularly any one of examples 7-9, wherein the vertical strut at the first location of the frame comprises two or more openings through which the actuator member extends, and wherein the opening of the two or more openings positioned closest to the outflow end of the frame is larger than the other openings of the two or more openings.

Example 11. The prosthetic heart valve of any example herein, particularly any one of examples 7-10, wherein the actuator member is coupled to one or more of the vertical struts via a knot.

Example 12. The prosthetic heart valve of any example herein, particularly any one of examples 1-11, wherein the actuator member comprises one or more of a suture, string, wire, cord, and/or cable.

Example 13. The prosthetic heart valve of any example herein, particularly any one of examples 1-12, wherein the actuator member is configured to be pulled to radially expand and/or lock the prosthetic heart valve.

Example 14. The prosthetic heart valve of any example herein, particularly any one of examples 1-13, wherein the locking element comprises a spring tooth that is naturally biased to provide a compressive force to the actuator member to prevent movement of the actuator member relative to the locking element in the distal direction.

Example 15. The prosthetic heart valve of any example herein, particularly example 14, wherein the locking element is formed in an axially extending opening in a strut of the frame.

Example 16. A prosthetic heart valve, comprising:
an annular frame comprising an inflow end and an outflow end, wherein the frame is radially compressible and expandable between a radially compressed state and a radially expanded state, and wherein the frame is configured to radially self-expand to at least a partially expanded state when a restraining mechanism configured to hold the frame in the radially compressed state is loosened;
a skirt assembly that extends circumferentially around the frame, wherein the skirt assembly comprises a sleeve that is configured to receive the restraining mechanism;
an actuator member coupled to the frame at first and second axially spaced locations, wherein the actuator member is configured to apply a proximally directed force to the frame to radially expand and/or lock the prosthetic heart valve; and
a locking element coupled to the frame at the second location, wherein the locking element is configured to prevent radial compression of the valve when the actuator member is in a taut state by allowing the actuator member to slide in only a proximal direction relative to the frame and by continuously locking the actuator member to prevent the actuator member from sliding in an opposite distal direction past the locking element.

Example 17. The prosthetic heart valve of any example herein, particularly example 16, wherein the annular frame comprises a first vertical strut at the first location of the frame and a second vertical strut at the second location of the frame, and wherein the actuator member is coupled to, and extends between, the first and second vertical struts.

Example 18. The prosthetic heart valve of any example herein, particularly example 17, wherein the first vertical strut and/or the second vertical strut comprise one or more openings that are axially spaced from one another, and wherein the actuator member extends through the one or more openings in the first vertical strut and/or the second vertical strut.

Example 19. The prosthetic heart valve of any example herein, particularly example 18, wherein the actuator member weaves in and out of the openings in the first vertical strut and is tied in a knot at an inflow apex of the first vertical strut to couple the actuator member to the first vertical strut.

Example 20. The prosthetic heart valve of any example herein, particularly example 18, wherein the actuator member weaves in and out of the openings in the first vertical strut, folds over an inflow apex of the first vertical strut, doubles back through the openings in the first vertical strut in a crisscrossing pattern, and is tied in a knot at an end of the first vertical strut opposite the inflow apex to couple the actuator member to the first vertical strut.

Example 21. The prosthetic heart valve of any example herein, particularly any one of examples 18-20, wherein the actuator member weaves in and out of the openings in the second vertical strut.

Example 22. The prosthetic heart valve of any example herein, particularly any one of examples 18-21, wherein the second vertical strut comprises two or more of the openings, and wherein the one of the two or more openings positioned closest to the outflow end of the valve is larger than the other openings.

Example 23. The prosthetic heart valve of any example herein, particularly any one of examples 18-22, wherein the frame and the actuator member frictionally engage one another in regions where the actuator member extends through the openings in the frame, and wherein the frictional engagement between the frame and the actuator member helps prevent radial compression of the frame.

Example 24. The prosthetic heart valve of any example herein, particularly any one of examples 16-23, wherein the locking element comprises a spring tooth that is configured to apply a compressive force to the actuator member to prevent the actuator member from moving in the distal direction relative to the locking element.

Example 25. The prosthetic heart valve of any example herein, particularly any one of examples 16-24, wherein the locking element is coupled to an outflow apex of the frame.

Example 26. The prosthetic heart valve of any example herein, particularly any one of examples 16-25, wherein the actuator member comprises one or more of a suture, string, wire, cord, and/or cable.

Example 27. The prosthetic heart valve of any example herein, particularly any one of examples 16-26, wherein the frame is configured to radially self-expand to a partially radially expanded state, and wherein the actuator member is configured to be pulled in the proximal direction to further radially self-expand the frame to the radially expanded state.

Example 28. The prosthetic heart valve of any example herein, particularly any one of examples 16-26, wherein the frame is configured to radially self-expand to the radially expanded state.

Example 29. The prosthetic heart valve of any example herein, particularly any one of examples 27 or 28, wherein the actuator member is configured to be pulled in the proximal direction to lock the valve is the radially expanded state.

Example 30. The prosthetic heart valve of any example herein, particularly any one of examples 16-29, wherein locking element is configured to hold the actuator member as the frame radially self-expands and axially foreshortens such that slack develops in the actuator member as the frame radially self-expands.

Example 31. The prosthetic heart valve of any example herein, particularly any one of examples 16-30, wherein when the actuator member is in a loose state, the frame is configured to be radially compressed by only tightening the restraining mechanism.

Example 32. The prosthetic heart valve of any example herein, particularly any one of examples 16-31, wherein the first location of the frame is an inflow end portion of the frame and wherein the second location is an outflow end portion of the frame.

Example 33. A prosthetic heart valve, comprising:
an annular frame comprising an inflow end and an outflow end, wherein the frame is radially compressible and expandable between a radially compressed state and a radially expanded state, wherein the frame is configured to self-expand from the radially compressed state to at least a partially expanded state;
a tension member coupled to the frame at first and second axially spaced locations along the frame; and
a locking element configured to engage the tension member at the second location and configured to allow the tension member to be pulled proximally relative to the locking element and resist distal movement of the tension member relative to the locking element, wherein the locking element engages the tension member such that slack is present in the tension member when the frame self-expands to at least the partially expanded state and application of a pulling force to the tension member is effective to remove the slack from the tension member to retain the frame in the at least partially expanded state.

Example 34. The prosthetic heart valve of any example herein, particularly example 33, wherein the frame comprises a first vertical strut at the first location and a second vertical strut at the second location, and wherein the first vertical strut is included at an inflow end portion of the frame and the second vertical strut is included at an outflow end portion of the frame.

Example 35. The prosthetic heart valve of any example herein, particularly example 34, wherein the first vertical strut and/or the second vertical strut comprise one or more radially extending and axially spaced openings through which the tension member is configured to extend.

Example 36. The prosthetic heart valve of any example herein, particularly example 35, wherein the tension member weaves in and out of the one or more radially extending and axially spaced openings in an alternating manner.

Example 37. The prosthetic heart valve of any example herein, particularly example 35, wherein the tension member extends through the one or more radially extending and axially spaced openings in a crisscross manner.

Example 38. The prosthetic heart valve of any example herein, particularly any one of examples 35-37, wherein an opening of the one or more radially extending and axially spaced openings positioned closest to an inflow end of the second vertical strut is wider than the other radially extending and axially spaced openings of the second vertical strut.

Example 39. The prosthetic heart valve of any example herein, particularly any one of examples 34-38, wherein the first vertical strut and/or the second vertical strut comprise an axially extending opening at an apex through which the tension member is configured to extend.

Example 40. The prosthetic heart valve of any example herein, particularly any one of examples 34-39, wherein the locking element is included at an outflow apex of the second vertical strut.

Example 41. The prosthetic heart valve of any example herein, particularly any one of examples 34-40, wherein the tension member is tied in a knot at the first vertical strut to couple the tension member to the first vertical strut.

Example 42. The prosthetic heart valve of any example herein, particularly any one of examples 32-41, wherein the locking element comprises a spring tooth.

Example 43. The prosthetic heart valve of any example herein, particularly any one of examples 32-42, wherein the tension member comprises one or more of a suture, string, wire, cord, and/or cable.

Example 44. The prosthetic heart valve of any example herein, particularly any one of examples 32-43, wherein, when slack is present in the tension member, the frame is configured to be radially compressed without disengaging the locking element.

Example 45. A method comprising:
loosening a restraining mechanism of a prosthetic heart valve to allow the prosthetic heart valve to radially self-expand; and
pulling an actuator member of an expansion and locking assembly of the prosthetic heart valve a first distance to tighten a locking element of the expansion and locking assembly from a loose state to a taut state to lock the prosthetic heart valve and prevent the prosthetic heart valve from radially compressing.

Example 46. The method of any example herein, particularly example 45, comprising before pulling the actuator member, but after loosening the restraining mechanism, radially re-compressing the prosthetic heart valve without releasing or disengaging a locking element of the expansion and locking assembly by only tightening the restraining mechanism.

Example 47. The method of any example herein, particularly example 46, wherein the locking element is configured to relax to the loose state as the prosthetic heart valve radially self-expands and is configured to tighten back towards the taut state as the prosthetic heart valve is re-compressed.

Example 48. The method of any example herein, particularly any one of examples 45-47, wherein the expansion and locking assembly is configured to continuously lock the actuator member to prevent the prosthetic heart valve from radially compressing once the actuator member is pulled to the taut state.

Example 49. The method of any example herein, particularly any one of examples 45-48, further comprising pulling the actuator member a second distance after pulling it the first distance to mechanically radially expand the prosthetic heart valve to a fully radially expanded state.

Example 50. The method of any example herein, particularly any one of examples 45-49, further comprising providing a distally directed force to the prosthetic heart valve that is opposite the pulling force via one or more support members that are configured to abut an outflow end of the prosthetic heart valve to help mechanically radially expand the prosthetic heart valve and/or to hold the prosthetic heart valve in place while radially expanding and locking the prosthetic heart valve.

Example 51. A prosthetic heart valve comprising:
an annular frame comprising an inflow end and an outflow end, wherein the frame is radially compressible and expandable between a radially compressed state and a radially expanded state, and wherein the annular frame further comprises:
   a first vertical strut at an inflow end portion of the frame; and
   a second vertical strut at an outflow end portion of the frame, wherein the first vertical strut and/or the second vertical comprise one or more axially spaced openings that extend radially through the frame;
a tension member coupled to the first vertical strut and the second vertical strut, wherein the tension member extends through the one or more axially spaced openings in the first vertical strut and/or the second vertical strut; and
a locking element configured to engage the tension member at the second vertical strut and configured to allow the tension member to be pulled proximally relative to the locking element and resist distal movement of the tension member relative to the locking element.

Example 52. The prosthetic heart valve of any example herein, particularly example 51, wherein the second vertical strut further comprises another opening at an outflow end of the second vertical strut that extends axially through the outflow end of the second vertical strut, and wherein the tension member extends through this axially extending opening at the outflow end of the second vertical strut.

Example 53. The prosthetic heart valve of any example herein, particularly any one of examples 51 or 52, wherein the tension member weaves in and out of the axially spaced openings of the second vertical strut in an alternating manner.

Example 54. The prosthetic heart valve of any example herein, particularly any one of examples 51-53, wherein the tension member extends through the axially spaced openings of the first vertical strut in a crisscross manner.

Example 55. The prosthetic heart valve of any example herein, particularly any one of examples 51-54, wherein the tension member comprises one or more of a suture, string, wire, cord, and/or cable.

Example 56. The prosthetic heart valve of any example herein, particularly any one of examples 51-55, wherein the locking element comprises a spring tooth.

Example 57. The prosthetic heart valve of any example herein, particularly any one of examples 51-56, wherein an opening of the one or more axially spaced openings of the second vertical strut positioned closest to an outflow end of the second vertical strut is larger than the other axially spaced openings of the second vertical strut.

Example 58. The prosthetic heart valve of any example herein, particularly any one of examples 51-57, wherein the valve is not self-expanding and is configured to be mechanically radially expanded from the radially compressed state to the radially expanded state and locked in the radially expanded state by pulling the tension member proximally.

Example 59. The prosthetic heart valve of any example herein, particularly any one of examples 51-57, wherein the prosthetic heart valve is fully self-expanding and is configured to radially self-expand to the radially expanded state when released from a restraining mechanism, and wherein the prosthetic heart valve is configured to be locked in the radially expanded state by pulling the tension member proximally.

Example 60. The prosthetic heart valve of any example herein, particularly any one of examples 51-57, wherein the prosthetic heart valve is partially self-expanding and is configured to radially self-expand to a partially radially expanded state when released from a restraining mechanism, wherein the partially radially expanded state is a state between the radially compressed state and the radially expanded state, and wherein the prosthetic heart valve is configured to be radially expanded from the partially radially expanded state to the radially expanded state and locked in the radially expanded state by pulling the tension member proximally.

Example 61. The prosthetic heart valve of any example herein, particularly any one of examples 51-60, further comprising an outer skirt coupled to an outer surface of the frame, the outer skirt comprising a sleeve for receiving a/the restraining mechanism, wherein the restraining mechanism is configured to selectively hold the prosthetic heart valve in the radially compressed state.

In view of the many possible examples to which the principles of the disclosed invention can be applied, it should be recognized that the illustrated examples are only preferred examples of the invention and should not be taken as limiting the scope of the invention. Rather, the scope of the invention is defined by the following claims. We therefore claim as our invention all that comes within the scope and spirit of these claims.

1. A prosthetic heart valve, comprising:
an annular frame comprising an inflow end and an outflow end, wherein the frame is radially compressible and expandable between a radially compressed state and a radially expanded state, and wherein the frame is configured to radially self-expand from the radially compressed state to at least a partially radially expanded state;
an actuator member coupled to the frame at first and second axially spaced locations, wherein the actuator member is configured to apply a proximally directed force to the frame to radially expand and/or lock the prosthetic heart valve; and
a locking element coupled to the frame at the second location, wherein the locking element is configured to allow the actuator member to slide in only a proximal direction relative to the frame and to continuously engage the actuator member to prevent the actuator member from sliding in an opposite distal direction past the locking element.

2. The prosthetic heart valve of claim 1, wherein the prosthetic heart valve is fully self-expanding and is configured to radially self-expand to the radially expanded state when released from a restraining mechanism.

3. The prosthetic heart valve of claim 1, wherein the prosthetic heart valve is partially self-expanding and is configured to radially self-expand to a partially radially expanded state when released from a restraining mechanism, wherein the partially radially expanded state is a state between the radially compressed state and the radially expanded state.

4. The prosthetic heart valve of claim 2, wherein the locking element is configured to continuously engage the actuator member while the prosthetic heart valve radially self-expands such that slack develops in the actuator member between the first and second locations as the valve axially foreshortens during the radial expansion.

5. The prosthetic heart valve of claim 3, wherein the actuator member is configured to radially expand the valve from the partially radially expanded state to the radially expanded state.

6. The prosthetic heart valve of claim 1, wherein the locking element is integrally formed in the frame.

7. The prosthetic heart valve of claim 1, wherein the frame comprises vertical struts at the first and second locations of the frame, and wherein the actuator member extends through one or more openings in one or more of the vertical struts.

8. The prosthetic heart valve of claim 7, wherein the actuator member weaves in and out of the openings in one or more of the vertical struts.

9. The prosthetic heart valve of claim 7, wherein the actuator member crisscrosses through the openings in one or more of the vertical struts.

10. The prosthetic heart valve of claim 7, wherein the vertical strut at the first location of the frame comprises two or more openings through which the actuator member extends, and wherein the openings of the two or more openings positioned closest to the outflow end of the frame is larger than the other openings of the two or more openings.

11. The prosthetic heart valve of claim 7, wherein the actuator member is coupled to one or more of the vertical struts via a knot.

12. The prosthetic heart valve of claim 1, wherein the actuator member comprises one or more of a suture, string, wire, cord, and/or cable.

13. The prosthetic heart valve of claim 1, wherein the actuator member is configured to be pulled to radially expand and/or lock the prosthetic heart valve.

14. The prosthetic heart valve of claim 1, wherein the locking element comprises a spring tooth that is naturally biased to provide a compressive force to the actuator member to prevent movement of the actuator member relative to the locking element in the distal direction.

15. The prosthetic heart valve of claim 14, wherein the locking element is formed in an axially extending opening in a strut of the frame.

16. A prosthetic heart valve, comprising:
an annular frame comprising an inflow end and an outflow end, wherein the frame is radially compressible and expandable between a radially compressed state and a radially expanded state, and wherein the frame is configured to radially self-expand to at least a partially expanded state when a restraining mechanism configured to hold the frame in the radially compressed state is loosened;
a skirt assembly that extends circumferentially around the frame, wherein the skirt assembly comprises a sleeve that is configured to receive the restraining mechanism;
an actuator member coupled to the frame at first and second axially spaced locations, wherein the actuator member is configured to apply a proximally directed force to the frame to radially expand and/or lock the prosthetic heart valve; and
a locking element coupled to the frame at the second location, wherein the locking element is configured to prevent radial compression of the valve when the actuator member is in a taut state by allowing the actuator member to slide in only a proximal direction relative to the frame and by continuously locking the actuator member to prevent the actuator member from sliding in an opposite distal direction past the locking element.

17. The prosthetic heart valve of claim 16, wherein the locking element is coupled to an outflow apex of the frame.

18. The prosthetic heart valve of claim 16, wherein the actuator member comprises one or more of a suture, string, wire, cord, and/or cable.

19. The prosthetic heart valve of claim 16, wherein the frame is configured to radially self-expand to a partially radially expanded state, and wherein the actuator member is configured to be pulled in the proximal direction to further radially self-expand the frame to the radially expanded state.

20. The prosthetic heart valve of claim 16, wherein the frame is configured to radially self-expand to the radially expanded state.

21. The prosthetic heart valve of claim 19, wherein the actuator member is configured to be pulled in the proximal direction to lock the valve is the radially expanded state.

22. A prosthetic heart valve, comprising:
an annular frame comprising an inflow end and an outflow end, wherein the frame is radially compressible and expandable between a radially compressed state and a radially expanded state, wherein the frame is configured to self-expand from the radially compressed state to at least a partially expanded state;
a tension member coupled to the frame at first and second axially spaced locations along the frame; and
a locking element configured to engage the tension member at the second location and configured to allow the tension member to be pulled proximally relative to the locking element and resist distal movement of the tension member relative to the locking element, wherein the locking element engages the tension member such that slack is present in the tension member when the frame self-expands to at least the partially expanded state and application of a pulling force to the tension member is effective to remove the slack from the tension member to retain the frame in the at least partially expanded state.

23. The prosthetic heart valve of claim 22, wherein the locking element comprises a spring tooth.

24. The prosthetic heart valve of claim 22, wherein the tension member comprises one or more of a suture, string, wire, cord, and/or cable.

25. The prosthetic heart valve of claim 22, wherein, when slack is present in the tension member, the frame is configured to be radially compressed without disengaging the locking element.
